(19) United States
(12) Patent Application Publication
(10) Pub. No.: US 2009/0287157 A1
(43) Pub. Date: Nov. 19, 2009

(54) FISTULA CATHETER AND FISTULA CATHETER SET

(75) Inventor: Shigeaki Funamura, Fukuroi-shi (JP)

Correspondence Address: TYCO HEALTHCARE - EDWARD S. JARMOLOWICZ, 15 HAMPSHIRE STREET, MANSFIELD, MA, 02048, US

(73) Assignee: TYCO HEALTHCARE GROUP LP, Mansfield, MA (US)

(21) Appl. No.: 12/464,720

(22) Filed: May 12, 2009

(30) Foreign Application Priority Data
May 14, 2008 (JP) 2008-126658

(51) Int. Cl. A61M 25/04

(52) U.S. Cl. 604/175

(57) ABSTRACT

A fistula catheter with a simple structure and with which it is possible to suppress irritation of the wall of the alimentary canal, and a fistula catheter set. A fistula catheter ‘A’ which is fitted in fistulae formed in a patient's abdominal wall and stomach wall consists of an external retention part which is disposed on the abdominal wall surface side of the fistula, and a tubular part. The tubular part is arranged such that the base end part thereof links with the external retention part, and the tip end part thereof extends from the fistulae into the stomach. The tubular part is formed as a flexible helix. Furthermore, the tip end part of the tubular part projects to the inner periphery of the helix. In addition, the angle between the central axis ‘a’ of the external retention part and the central axis ‘b’ of the helical tubular part is set to be smaller than a right angle.

### FIELD OF THE INVENTION

The present invention generally relates to a fistula catheter which is used to supply fluids such as liquid nutrients to the alimentary canal of a patient, and to a fistula catheter set.

### BACKGROUND OF THE INVENTION

It is a conventional practice to use fistula catheters to supply fluids such as liquid nutrients to patients who have a reduced capacity to feed themselves orally due to reasons such as advanced age, or illness. A fistula catheter typically consists of a tubular part which is inserted into a fistula formed in the abdominal wall of the patient, and an external retention part which is fitted to a base end part of the tubular part and is disposed on the skin surface side of the abdominal wall. After the fistula catheter has been fitted to the fistula, and the stomach wall and the abdominal wall have been fixed, it is necessary to provide a variable length of the tubular part to maintain a state in which the fistula is not pressured. This is done to prevent the formation of ulcers and to prevent the external retention part from becoming embedded inside the body at the fistula due to changes in the length of the gastric fistula.

Accordingly, the prior art describes an instrument in which the length between the external retention part and the tip end of the tubular part can be adjusted by forming the tubular part as a helix so as to make it extendible (see Japanese Patent Application PA08-096, for example). This fistula catheter (referred to by PA08-096 as an indwelling catheter) consists of an external retention part (flange) and a flexible tubular part (tube) inside which a lumen is formed. The external retention part consists of a flat base part formed with an opening which is continuous with the lumen of the tubular part, and a hinged cover for the base part. The cover is provided with a protrusion enabling the opening of the base part to be closed off.

Furthermore, the tubular part consists of a short linear portion positioned on the external retention part interfacing side, a helical portion which links with the linear portion, a long linear portion which links with the helical portion, and a tip-end “pigtail” of the long linear portion which bends so as to describe a circle. When the tubular part is inserted into the patient's gastric fistula, the helical portion deforms, returning to its original shape when it enters inside the gastric fistula, and therefore it is possible to prevent it from being removed from the patient, and also it is possible to maintain suitable spacing between the abdominal wall and the stomach.

However, with the fistula catheter described above, the tubular part consists of four different portions (short linear, helical, long linear and pigtail) which have differing shapes and lengths, and the shapes are complex and difficult to produce. Furthermore, the linear portion linking the helical portion and the pigtail is relatively long, and therefore the tubular part becomes long overall. As a result, the tip end of the tubular portion is likely to irritate the stomach wall.

### SUMMARY OF THE INVENTION

In general, this invention is directed to a fistula catheter fitted in a fistula formed in an abdominal wall and a wall of an alimentary canal of a patient. The fistula catheter is provided with a flow channel for supplying fluid from outside the patient's body for ingestion to the alimentary canal of the patient. The fistula catheter comprises an external retention part disposed on an abdominal wall surface side of the fistula, and a flexible tubular part formed as a helix. The flexible tubular part further comprises a base end part that links with the external retention part, and a tip end part that extends from the fistula into the alimentary canal.

Other objects and features will be in part apparent and in part pointed out hereinafter.

### BRIEF DESCRIPTION OF THE DRAWINGS

FIG. 1 is an oblique view of a fistula catheter according to a first mode of embodiment of the present invention in which a cover part is open.

FIG. 2 is a plan view of the fistula catheter of FIG. 1.

FIG. 3 is an oblique view of the fistula catheter shown in FIG. 1 in which the cover part is closed.

FIG. 4 is a plan view of the fistula catheter shown in FIG. 3.

FIG. 5 is a plan view of a state in which an axial direction of a tubular part of the fistula catheter is vertical.

FIG. 6 is a front view showing an extension instrument according to an embodiment of the invention.

FIG. 7 is a front view of the extension instrument as fitted to the fistula catheter.

FIG. 8 is a partial view in section of the fistula catheter positioned above the abdomen to which the extension instrument has been fitted.

FIG. 9 is a partial view in section of the fistula catheter fitted in the fistula to which the extension instrument has been fitted.

FIG. 10 is a partial view in section of a state in which the extension instrument has been removed from the fistula catheter in FIG. 9.

FIG. 11 is an oblique view of a fistula catheter according to the second mode of embodiment of the present invention.

### EXPLANATION OF REFERENCE CHARACTERS

10 . . . external retention part;
14 . . . non-return valve;
20 . . . tubular part;
21 . . . base end part;
22 . . . tip end part;
25 . . . extension part;
26 . . . grip part;
27 . . . insertion part;
31, 32 . . . belt;
A, B . . . fistula catheter;
S . . . stomach;
AW . . . abdominal wall;
SW . . . stomach wall;
AH, SH . . . fistula; and
a, b . . . central axis.

Corresponding reference characters indicate corresponding parts throughout the drawings.

### DESCRIPTION OF THE PREFERRED EMBODIMENTS

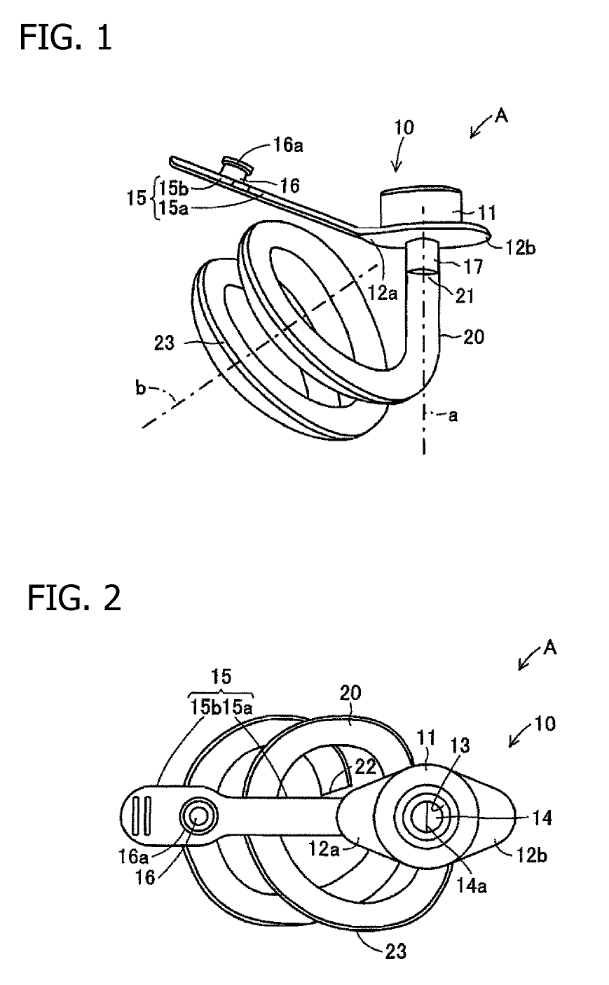

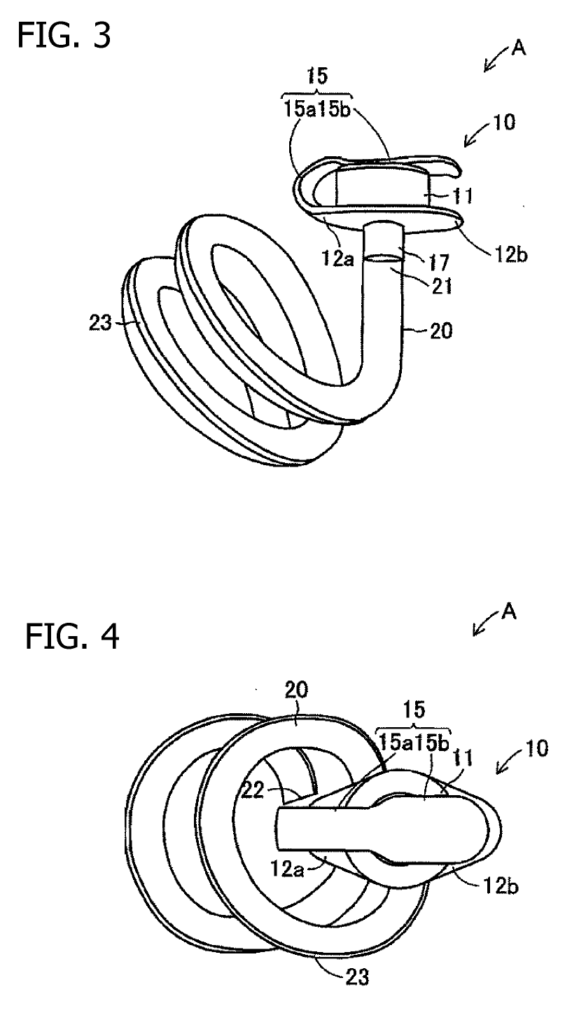

A first embodiment of the present invention is described below. FIGS. 1-4 show a fistula catheter A according to the first embodiment. The fistula catheter A consists of an external retention part 10 made of polyurethane, and a tubular part 20 which is linked to the center of a lower end surface of the external retention part 10. The upper side of FIGS. 1 and 3 illustrates the upper part of the fistula catheter A, and the lower side of FIGS. 1 and 3 illustrates the lower part of the fistula catheter A.

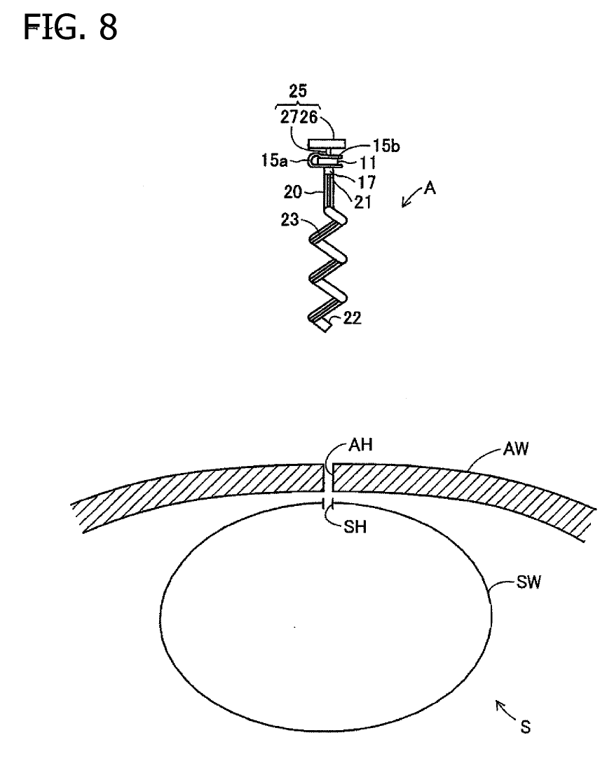

The main body portion of the external retention part 10 comprises an insertion opening 11 which is annular and somewhat thick, and projecting pieces 12a, 12b which project on both sides of the lower end part of the insertion opening 11. The outline of the portion including said projecting pieces 12a, 12b and insertion opening 11 is substantially elliptical when seen from above. The function of the projecting pieces 12a, 12b is to prevent the fistula catheter A from being pulled inside the stomach S (see FIGS. 8 to 10). A non-return valve 14 having a slit 14a formed in the center is then provided on the inner peripheral surface of an insertion hole 13 passing vertically through and formed in the centre of the insertion opening 11. Furthermore, an engaging groove part is formed around the circumference at the upper part of the non-return valve 14 on the inner peripheral surface of the insertion hole 13 (not shown).

A cover part 15 for closing off the insertion hole 13 of the insertion opening 11 is then joined to the tip end part of the projecting piece 12a. The cover part 15 consists of an elongated strip-shaped joining part 15a which is joined to the tip end part of the projecting piece 12a, and a broad part 15b which is formed at the tip end of the strip-shaped joining part 15a. The broad part 15b is wider and shorter than the strip-shaped joining part 15a. The strip-shaped joining part 15a is flexible and it can bend vertically or through sharp angles, with the joining part having the projecting piece 12a at the center. A cylindrical column part 16 which is shorter in the axial direction is provided on the broad part 15b.

The column part 16 is set to be opposite the insertion hole 13 when the strip-shaped joining part 15a is bent to position the broad part 15b above the insertion opening 11, and is provided on a portion of the broad part 15b on the strip-shaped joining part 15a side. The column part 16 fits into the insertion hole 13, and an annular protrusion 16a which can detachably engage with the engaging groove part formed on the inner peripheral surface of the insertion hole 13 is provided along the periphery on the inner peripheral surface of the insertion hole 13. Accordingly, the engaging groove part and the annular protrusion 16a can be caused to engage by bending the strip-shaped linking part 15a so as to vertically rotate it and pushing the column part 16 into the insertion hole 13.

In this manner, the insertion hole 13 of the insertion opening 11 can be closed off in a liquid-tight and airtight manner. In this case, the fistula catheter A reaches the state shown in FIGS. 3 and 4. Furthermore, it is possible to open the insertion hole 13 of the insertion opening 11 by pulling the broad part 15b to release the engagement between the column part 16 and the insertion hole 13. In this case, the fistula catheter A reaches the state shown in FIGS. 1 and 2. A cylindrical joining part 17 provided with a hole (not shown) in communication with the insertion hole 13 is then formed on the lower end surface of the main body portion of the external retention part 10.

The tubular part 20 consists of an elongate cylindrical member formed as a helix, and an insertion hole (not shown) is formed therein in order to allow the passage of fluids such as nutrients or liquid food. The base end part 21 of the tubular part 20 is connected to the joining part 17, and the insertion hole of the tubular part 20 is in communication with the insertion hole 13 of the insertion opening 11, by way of the hole in the joining part 17. Furthermore, the tubular part 20 is joined such that it lies obliquely to the external retention part 10, and the angle between the central axis ‘a’ of the external retention part 10 (an imaginary line passing through the centre of the insertion hole 13 of the insertion opening 11 and the hole of the joining part 17) and the central axis ‘b’ of the tubular part 20 (the imaginary line passing through the centre of the helix of the tubular part 20) is set to be substantially 45°.

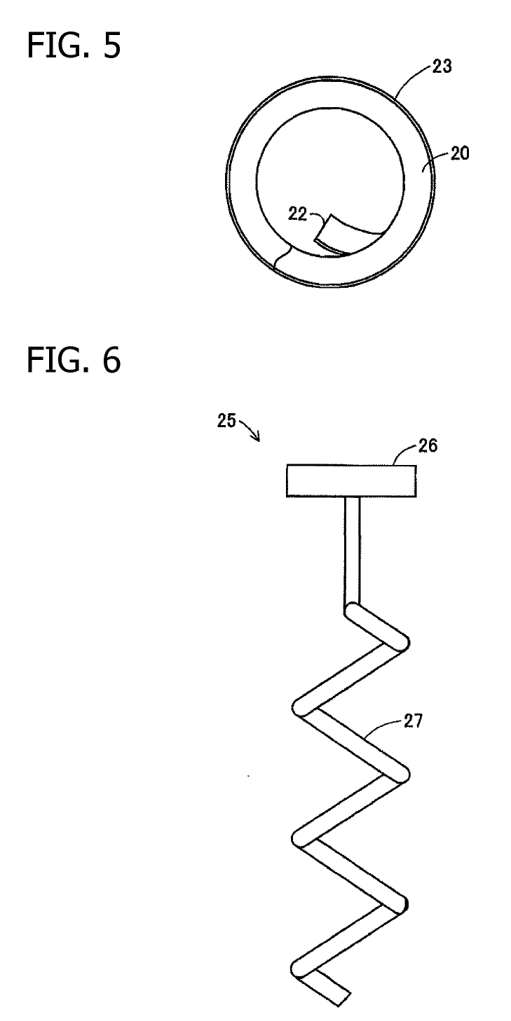

Furthermore, FIG. 5 shows the tubular part 20 seen vertically from above along the central axis b, where the external retention part 10 has been removed from the fistula catheter A, and as shown in FIG. 5, the opening at the tip end part 22 of the tubular part 20 is oriented towards the inside of the helix so as to project to the inner periphery of the helix. A contrasting line 23 running from the base end part 21 to the tip end part 22 is then formed on a specific portion of the outer peripheral surface of the tubular part 20. This line 23 is made of a specific material which is impermeable to X-rays, and its position can be ascertained by capturing images when irradiated with X-rays. Furthermore, the tubular part 20 is flexible, and it extends along the direction of the central axis b by being pulled between the base end part 21 and the tip end part 22.

FIG. 6 shows an extension instrument 25 which is used when the fistula catheter A is fitted into the patient's body. This extension instrument 25 is made of a resin more rigid than that of fistula catheter A such as polypropylene, polyurethane, silicone, polycarbonate, polyethylene or nylon, or a metal such as stainless steel. The extension instrument 25 is configured by joining the base end part of a helical rod-shaped insertion part 27 (which is narrower and longer than a straight rod-shaped grip part 26, which runs horizontally) to the central part of the grip part 26. Moreover, the surface of the insertion part 27 may be coated with silicone, or be subjected to texturing so as to improve its insertability when it is inserted into the fistula catheter A. Furthermore, the insertion part 27 is formed of a thickness such that it can pass from the insertion hole 13 of the fistula catheter A through the insertion hole in the tubular part 20. The helix of the insertion part 27 is formed so as to be shorter in the radial direction and longer in the axial direction than the helix of the tubular part 20.

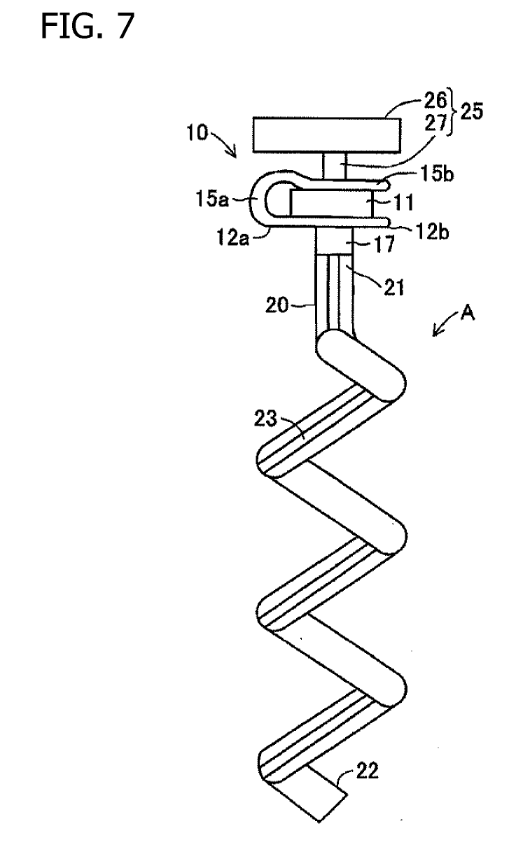

In this configuration, when the insertion part 27 of the extension instrument 25 is inserted from the insertion hole 13 and enters the fistula catheter A (as shown in FIG. 7), the tubular part 20 of the fistula catheter A becomes a helix which extends to follow the shape of the insertion part 27 of the extension instrument 25. When the insertion part 27 of the extension instrument 25 is inserted into the fistula catheter A, the insertion part 27 readily enters the fistula catheter A, by pushing of the extension instrument 25 while it is rotated with respect to the fistula catheter A. Furthermore, when the extension instrument 25 is withdrawn from the fistula catheter A, the extension instrument 25 can be simply withdrawn by pulling the extension instrument 25 while rotating it in the direction opposite to the abovementioned direction of rotation. The fistula catheter set according to the present invention consists of said extension instrument 25 and the abovementioned fistula catheter A.

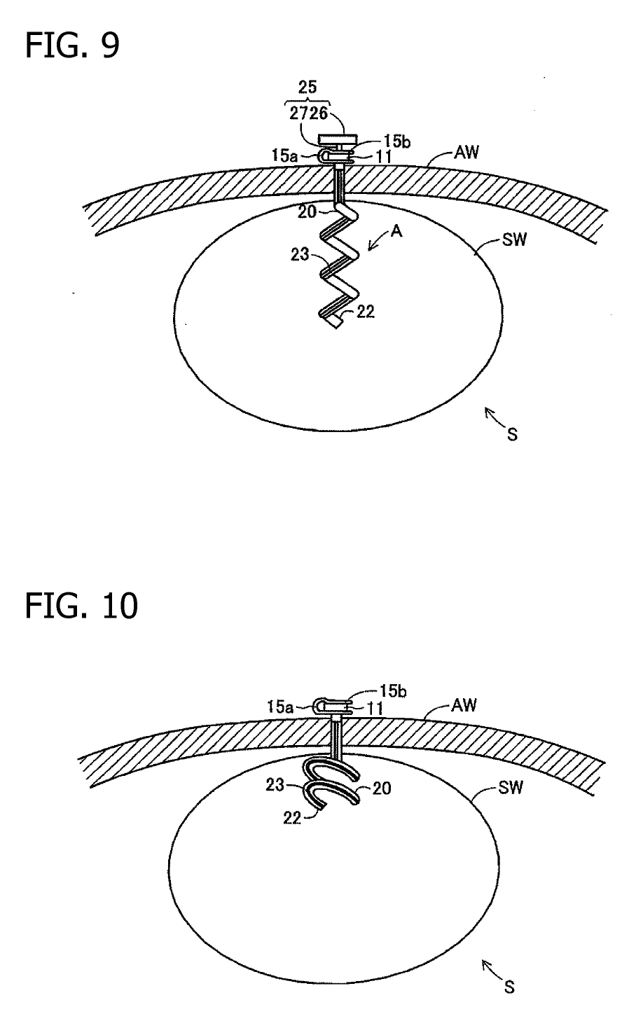

When the fistula catheter A is fitted into the patient's fistula using this configuration, fistulae AH, SH are first of all formed in the patient's abdominal wall AW and stomach wall SW respectively, after which the extension instrument 25 is fitted to the fistula catheter A, as shown in FIG. 7. Next, the fistula catheter A to which the extension instrument 25 has been fitted is positioned above the patient's abdominal wall AW, as shown in FIG. 8, after which it is passed through the fistulae AH, SH. In this case, the fistula catheter A passes through the fistulae AH, SH together with the extension instrument 25 by being screwed in while being rotated. As shown in FIG. 9, once most of the tubular part 20 has entered the stomach S of the patient, the extension instrument 25 is withdrawn from the fistula catheter A.

In this manner, the shape of the tubular part 20 returns to its original helical shape, being relatively longer in the radial direction and shorter in the axial direction. In this case, the portion of the tubular part 20 which is positioned within the fistulae AH, SH extends substantially linearly, and the upper part of the helical portion is in contact with the stomach wall SW. As a result, the fistula catheter A is prevented from being withdrawn from the fistulae AH, SH, and it remains fixed in the patient's abdomen. Next, the insertion hole 13 of the insertion opening 11 is closed off using the cover part 15. This completes the fitting of the fistula catheter A to the patient. This state is then maintained for 1-2 days. During this time, bleeding from the fistulae AH, SH is stopped, and the abdominal wall AW and stomach wall SW are fixed.

When the patient ingests fluids such as liquid food or nutrients, the cover part 15 is removed from the insertion opening 11 to open the insertion hole 13, and a fluid supply tube (not shown) is connected to the insertion hole 13. In this state, fluid is introduced into the fluid supply tube from an end-part opening of the fluid supply tube. As a result, the fluid is supplied to the patient's stomach S from the fluid supply tube, via the insertion hole 13, insertion hole of the joining part 17, and the insertion hole of the tubular part 20. Furthermore, once the fluid has been ingested, the fluid supply tube is removed from the external retention part 10, and the insertion hole 13 is closed.

With this fistula catheter A as described above, the tubular part 20 is formed as a flexible and extendible helix. Consequently, the fistula catheter A is kept in a state in which it is shorter in the axial direction when no external force is applied to it, and when an extension force in the axial direction is applied to the tubular part 20, the tubular part 20 extends. Accordingly, the fistulae AH, SH are formed in the abdominal wall AW and stomach wall SW, and when the fistula catheter A is fitted to the fistulae AH, SH, it is simple to operate; after the fistula catheter A has been fixed in the fistulae AH, SH, the abdominal wall AW and the stomach wall SW are held by the external retention part 10 and a portion of the tubular part 20 which retains its helical shape, thereby maintaining a fixed positional relationship.

At this time, the tubular part 20 extends or contracts in accordance with the patient's body size and the positional relationship between the abdominal wall AW and the stomach wall SW, and therefore it is possible to use one size of fistula catheter A for all patients. Furthermore, it is possible to provide room for the length between the external retention part 10 and the portion of the tubular part 20 positioned on the inner surface of the stomach wall SW, in respect of the length of the fistulae AH, SH, and therefore it is possible to prevent the formation of ulcers. Also, when the fistula catheter A is fixed in the patient's fistulae AH, SH, there is no need to replace the fistula catheter A even if the condition of the patient changes. Furthermore, when the tubular part 20 has extended substantially linearly from its helical shape, it becomes longer in the axial direction, and therefore the fistula catheter A does not come out from the patient's body.

Furthermore, with the fistula catheter A according to this first embodiment, the angle between the central axis a of the external retention part 10 and the central axis b of the tubular part 20 is set to be substantially 45°. Consequently, when the fistula catheter A is fitted to the fistulae AH, SH, it is possible to shorten the length between the external retention part 10 and the tip end part 22 of the tubular part 20 in the direction along the central axis a of the external retention part 10, or the direction parallel to the central axis a of the external retention part 10. This makes it possible to prevent the tip end part 22 of the tubular part 20 from coming into contact with the portion opposite the portion of the stomach wall SW where the fistula SH is formed and from irritating the stomach wall SW.

Furthermore, a non-return valve 14 is provided in the insertion hole 13 of the fistula catheter A, and therefore it is possible to prevent fluid inside the stomach S from flowing back in the fistula catheter A, and from flowing outside the body. Furthermore, the fistula catheter set according to the present invention consists of the fistula catheter A and the extension instrument 25; hence, the operation to fit the fistula catheter A into the fistulae AH, SH is simplified. The insertion part 27 of the extension instrument 25 is formed as a helix whereof the length in the radial direction is shorter and the length in the axial direction is longer than that of the helix of the tubular part 20. Therefore when the fistula catheter A is fitted into the patient's fistulae AH, SH, the fistula catheter A and the extension instrument 25 can be inserted into the fistulae AH, SH while being rotated to screw them in, and the operation is simplified and insertion can be carried out more reliably.

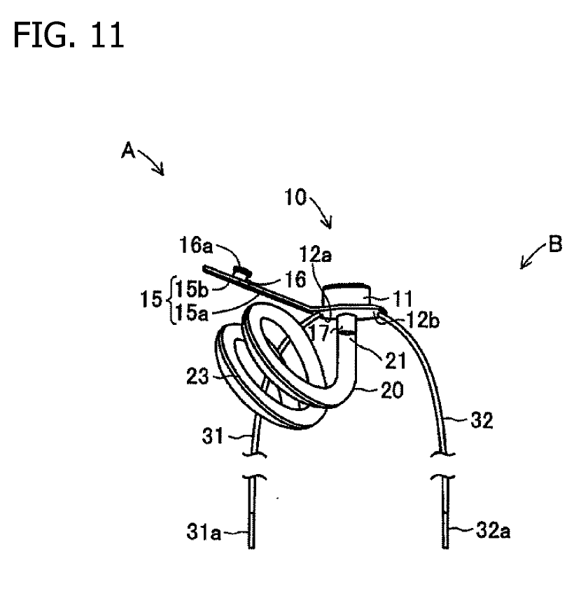

FIG. 11 shows a fistula catheter B according to a second embodiment of the present invention. The fistula catheter B has a configuration in which a pair of belts 31, 32 act as the fastening member and are joined to the lower part at the tip end edge of the projecting pieces 12a, 12b in the peripheral direction on the outer peripheral surface of the insertion opening 11 of the fistula catheter A described above. Surface fasteners 31a, 32a which are detachable from each other are then provided at the tip end part of the belts 31, 32. The belts 31, 32 have a length such that they can surround the trunk part of an adult of large constitution, and by changing the joining position of the surface fasteners 31a, 32a, it is possible to make them fit any patient, regardless of body size. The other structural components of this fistula catheter B are the same as those of the fistula catheter A of the first embodiment of the present invention. Accordingly, components which are the same bear the same reference numbers, and are not described further.

Furthermore, with the fistula catheter B, the abovementioned extension instrument 25 is used as the extension instrument and is part of the fistula catheter set. Therefore, when this fistula catheter B is fitted into the fistula of a patient, the same fitting operation is carried out as for the fistula catheter A described above, and the fistula catheter B is fixed in the fistulae AH, SH, after which the belts 31, 32 are wound around the patient's body, and the surface fasteners 31a, 32a are joined together. This makes it possible to even more reliably prevent the fistula catheter B from coming away from the patient's body. The other operational effects of this fistula catheter B and the fistula catheter set which is provided with the fistula catheter B are the same as the operational effects of the fistula catheter A and the fistula catheter set which is provided with the fistula catheter A which were described above.

The fistula catheter and the fistula catheter set according to the present invention are not limited to the modes of embodiment described above, and appropriate modifications may be made within the technical scope of the present invention. For example, in the various embodiments described above, the angle between the central axis a of the external retention part 10 and the central axis b of the tubular part 20 is set at 45°, but this angle is not limited to 45°, and it may be appropriately changed to any angle less than 90°. Furthermore, the central axis ‘a’ of the external retention part 10 and the central axis ‘b’ of the tubular part 20 may be coaxial and not provided obliquely. Furthermore, the tip end part 22 of the tubular part 20 need not project to the inner side of the helix, and it may be positioned on a portion along the periphery of the helix.

In addition, a pair of cords which can be joined or a pair of strip-shaped members provided with an engaging part and a part to be engaged, or similar, may be used instead of the belts 31, 32 as the fastening members. Furthermore, in the various embodiments described above, the non-return valve 14 is provided in the insertion hole 13 of the insertion opening 11, but said non-return valve 14 may be provided inside the tubular part 20. In addition, in the various embodiments described above, the insertion part 27 of the extension part 25 is helical, but said insertion part 27 may also be made linear.

Furthermore, in the various embodiments described above, the fistula catheters A, B are made of polyurethane, but a resin material such as polypropylene, silicone or polycarbonate may also be used as the material from which said fistula catheters A, B are made. In addition, in the various embodiments described above, the fistula catheters A, B are fixed in fistulae AH, SH formed in the patient's abdominal wall AW and stomach wall SW, but the fistula catheters A, B may also be fixed in fistulae formed in the patient's abdominal wall AW and intestines to supply fluid such as liquid food or nutrients to the intestines. Furthermore, gas for making the stomach or intestines swell for examination purposes and the like may also be contained in the fluid supplied to the stomach or intestines.

The present invention overcomes one or more deficiencies in the prior art by providing a fistula catheter with a simple structure and with which it is possible to reduce irritation of the wall of the alimentary canal, and a fistula catheter set.

Structural features of the present invention include a fistula catheter which is fitted in a fistula formed in a patient, and which is provided with a flow channel that supplies fluid from outside the patient's body for ingestion to the alimentary canal of the patient. The fistula catheter comprising an annular or tubular external retention part disposed on the abdominal wall outside the fistula, and a flexible tubular part arranged so that the base end part thereof links with the external retention part, and the tip end portion thereof extends from the fistula into the alimentary canal, with the tubular part being formed as a helix.

With the fistula catheter according to one embodiment of the present invention configured as described above, the helical tubular part is fitted to pass through the fistula and extend into the alimentary canal of the patient. The tubular part is flexible, and therefore the helical tubular part is extendible in an axial direction. Consequently, the fistula catheter normally maintains a state in which it is helically coiled and short in the axial direction, and when a force is applied to a specific portion of the tubular part in a direction away from the external retention part, the portion between that specific portion of the tubular part and the external retention part extends axially.

Accordingly, when the fistula catheter is fitted to the gastric fistula, the abdominal wall and the wall of the alimentary canal are held between the external retention part and a specific portion of the tubular part, thereby maintaining a fixed positional relationship. In this position, the tubular part extends or contracts in accordance with the patient's body size over time (e.g. the patient gains weight and the abdominal wall thickens, thereby increasing the length of the gastric fistula) and the positional relationship between the abdominal wall and the wall of the alimentary canal. Therefore, it is possible to use a single size of the fistula catheter for all patients, in spite of differences in patients' constitutions and other factors. In other words, the fistula catheter according to the present invention can function with abdominal walls and walls of alimentary canals of any thickness, and therefore eliminates the need to design fistula catheters of differing sizes.

Furthermore, since it is possible to vary the length between the external retention part and the portion of the tubular part positioned inside the inner wall of the alimentary canal in accordance with the length of the fistula, it is therefore possible to prevent the formation of ulcers. Also, when the fistula catheter is fitted in the patient's fistula, there is no need to replace the fistula catheter even if the nutritional state of the patient changes. The tubular part can extended substantially linearly from its helical shape, and therefore the fistula catheter does not come out from the patient's body under everyday conditions. In addition, the fistula catheter according to the present invention simply comprises the external retention part and the tubular part, thereby simplifying production and reducing costs.

Additional structural features of the fistula catheter according to an embodiment of the present invention include an angle between a central axis of the external retention part and a central axis of the helical tubular part that is set to be smaller than a right angle. By virtue of this feature, when the fistula catheter is fitted in the fistula, it is possible to shorten the length between the external retention part and the tip end part of the tubular part in the direction along or parallel to the central axis of the external retention part. Consequently, it is possible to prevent the tip end part of the tubular part from contacting and irritating the alimentary canal opposite to where the fistula is formed.

Additional structural features of the fistula catheter according to an embodiment of the present invention include projecting the tip end part of the tubular part into the inner periphery of the helix. This feature also prevents the tip end part of the tubular part from irritating the wall of the alimentary canal. As a result, the portion of the tubular part which is in contact with the wall of the alimentary canal constitutes a curved portion of the outer peripheral surface, thereby alleviating irritation of the wall of the alimentary canal.

Additional structural features of the fistula catheter according to an embodiment of the present invention include forming the tubular part as a cylindrical tube of substantially constant diameter from the base end side up to the tip end side. The helix of the tubular part defines an outer periphery and an inner periphery, where outer peripheral and inner peripheral diameters, as measured in a direction perpendicular to the central axis of the helix of the tubular part, are substantially the same along the central axis. It is possible, though, that the base end part and tip end part be positioned away from said outer periphery and inner periphery. As a result of this design, the tip end side of the tubular part does not narrow or widen relative to the tip.

Additional structural features of the fistula catheter according to an embodiment of the present invention include a strip-shaped or cord-shaped fastening member for fitting the external retention part is disposed on the abdominal wall body surface side of the fistula to the external retention part, or in the region thereof. In this manner, it is possible to reliably prevent the fistula catheter from being removed from the patient's body. Alternately, other similar fastening members such as an elongate cord, an extended strip-shaped body or a belt may be used, and may be fixed to the fistula catheter or detachable therefrom. Furthermore, the fastening member may be directly attached to the external retention part, or a fastening piece or the like may be formed, and it may be attached by way of said fastening piece or the like.

Additional structural features of the fistula catheter according to an embodiment of the present invention include a non-return valve for preventing reverse flow of the fluid inside the alimentary canal provided on a specific portion of the flow channel. In this manner, it is possible to prevent fluid inside the alimentary canal from flowing back through the fistula catheter to outside the body. The non-return valve may be provided in the external retention part, or it may be provided in the tubular part.

The structural features of the fistula catheter set according to the present invention lie in the fact that it consists of the fistula catheter disclosed in any one of the embodiments above, and an extension instrument provided with an insertion part. The insertion part comprises a member insertable into the fistula catheter and which deforms less readily than the fistula catheter. In this manner, when the fistula catheter is fitted into the patient's fistula, the fistula catheter does not readily deform due to the simultaneous insertion of the extension instrument into the fistula catheter, allowing easy insertion of the fistula catheter into the fistula. Furthermore, the extension instrument is provided with a grip part which is preferably grippable by the hand to operate the extension instrument.

Additional structural features of the fistula catheter set according to an embodiment of the present invention include forming the insertion part as a helix whereof the length of the helix in a radial direction is shorter and the length of the helix in the axial direction is longer than the helix of the tubular part. In this manner, when the fistula catheter is fitted into the patient's fistula, the extension instrument is insertable into the fistula catheter while being rotated. When the fistula catheter to which the extension instrument is fitted is inserted into the fistula, it is screwed in to allow insertion into the fistula upon rotation of the extension instrument. Furthermore, detaching the extension instrument from the fistula catheter is possible by rotating the extension instrument in the opposite direction to the abovementioned direction of rotation.

Having described the invention in detail, it will be apparent that modifications and variations are possible without departing from the scope of the invention defined in the appended claims.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions and methods without departing from the scope of the invention, it is intended that all matter contained in the above description and shown in the accompanying drawing[s] shall be interpreted as illustrative and not in a limiting sense.

What is claimed is:

1. A fistula catheter fitted in a fistula formed in an abdominal wall and a wall of an alimentary canal of a patient, said fistula catheter provided with a flow channel for supplying fluid from outside the patient's body for ingestion to the alimentary canal of the patient, said fistula catheter comprising:
an external retention part disposed on an abdominal wall surface side of the fistula; and
a flexible tubular part formed as a helix, said flexible tubular part comprising a base end part and a tip end part,
wherein the base end part links with the external retention part, and wherein the tip end part extends from the fistula into the alimentary canal.

2. A fistula catheter according to claim 1, wherein an angle formed between a central axis of the external retention part and a central axis of the helix of the flexible tubular part is smaller than a right angle.

3. A fistula catheter according to claim 2, wherein the tip end part projects into an inner periphery of the helix.

4. A fistula catheter according to claim 1, wherein a portion of the helix of the flexible tubular part from the base end part up to the tip end part has substantially the same diameter.

5. A fistula catheter according to claim 1, further comprising a fastening member for maintaining a state in which the external retention part is disposed on the abdominal wall surface side of the fistula, said fastening member being fitted to the external retention part or in the region thereof.

6. A fistula catheter according to claim 1, further comprising a non-return valve in the catheter flow channel for preventing the fluid inside the alimentary canal from flowing back outside the body.

7. A fistula catheter set comprising:
the fistula catheter of claim 1; and
an extension instrument provided with an insertion part, said insertion part comprising a member which can be inserted into the fistula catheter and which deforms less readily than the fistula catheter.

8. A fistula catheter set according to claim 7, wherein the insertion part is formed as a helix, wherein a length in the radial direction of the helix of the insertion part is shorter than a length in the radial direction of the helix of the tubular part, and wherein a length in the axial direction of the helix of the insertion part is longer than a length of the helix of the tubular part.

9. A fistula catheter according to claim 1, wherein the tip end part projects into an inner periphery of the helix.
